(19) United States
(12) Patent Application Publication
(10) Pub. No.: US 2016/0243061 A1
(43) Pub. Date: Aug. 25, 2016

(54) COMPOSITIONS COMPRISING CHOLINE AND DERIVATIVES THEREOF, USES THEREOF AND PROCESSES FOR THEIR PREPARATION

(71) Applicant: ENZYMOTEC LTD., Kfar Baruch (IL)

(72) Inventors: Asher WIDBERG, Haifa (IL); Ran NUMA, Haifa (IL); Gai BEN-DROR, Gita (IL); Fabiana BAR-YOSEPH, Haifa (IL)

(73) Assignee: ENZYMOTEC LTD., Kfar Baruch (IL)

(21) Appl. No.: 15/029,878

(22) PCT Filed: Oct. 21, 2014
(86) PCT No.: PCT/IL2014/050912

§ 371 Date: Apr. 15, 2016

Related U.S. Application Data
(60) Provisional application No. 61/893,307, filed on Oct. 21, 2013.
(60) Provisional application No. 61/893,308, filed on Oct. 21, 2013.

(51) Int. Cl. A61K 31/14; A61K 31/661; A61K 9/00

(52) U.S. Cl. CPC A61K 31/14; A61K 9/0095; A61K 31/661; A23L 1/296; A23L 1/30; A23V 2002/00

(57) ABSTRACT

The invention provides compositions comprising choline and water soluble derivatives thereof, processes for their preparation from natural sources and uses thereof.

### TECHNOLOGICAL FIELD

The invention provides composition comprising choline and water soluble derivatives thereof, processes for their preparation and uses thereof.

### BACKGROUND ART

References considered to be relevant as background to the presently disclosed subject matter are listed below:
1. Blusztajn J K. Choline, a vital amine. Science 1998; 281(5378)794-795.
2. Zeisel S H. Choline: an essential nutrient for humans. Nutrition 2000; 16(7-8):669-671.
3. Food and Nutrition Board, Institute of Medicine. Choline. In: Dietary Reference Intakes for Thiamin, Ribotlavin, Niacin, Vitamin B6, Vitamin B12, Pantothenic Acid, Biotin, and Choline. Washington. D.C.: National Academy Press, 1998:390-422.
4. Holmes-McNary M Q, Cheng W L, Mar M H, Fussell S, Zeisel S H. Choline and choline esters in human and rat milk and in infant formulas. Am J Clin Nutr. 1996 October; 64(4):572-6.
5. Holmes H C, Snodgrass G J, Iles R A. Changes in the choline content of human breast milk in the first 3 weeks after birth. Eur J Pediatr. 2000 March159(3):198-204.
6. Ilcol Y O, Ozbek R, Hamurtekin E, Ulus I H. Choline status in newborns, infants, children, breast-feeding women, breast-fed infants and human breast milk. J Nutr Biochem. 2005 Aug.; 16(8):489-99.
7. Kristine Y. Patterson, Seema A. Bhagwat, Juhi R. Williams, Juliette C. Howe, and Joanne M. Holden. USDA Database for the Choline Content of Common Foods. Release Two. Nutrient Data Laboratory Agricultural Research Service U.S. Department of Agriculture. January 2008.
8. Florián J. and Warshel. Phosphate Ester Hydrolysis in Aqueous Solution: Associative versus Dissociative Mechanisms. A. J. Phys. Chem. B. 1998, 102 (4), pp 719-734.
9. Kamerlin S C, Florián J, Warshel A. Associative versus dissociative mechanisms of phosphate monoester hydrolysis: on the interpretation of activation entropies. Chemphyschem. 2008 Aug. 25; 9(12):1767-73.
10. Vincent J B, Crowder M W, Averill B A. Hydrolysis of phosphate monoesters: a biological problem with multiple chemical solutions. Trends Biochem Sci. 1992 Mar.; 17(3):105-10.
11. U.S. Pat. No. 2,864,848 A1—Method of producing 1-alpha-glycerylphosphorylcholine.
12. WO 2007010892 A1—Novel phospholipid processing agent
13. Ozarda Yl, Cansev M, Ulus I H. Breast milk choline contents are associated with inflammatory status of breastfeeding women. J Hum Lact. 2014 May; 30(2):161-6.
14. Ozarda Yl, Cansev M, Ulus I H. Relations of human breastmilk choline content with maternal hormonal status. Breastfeed Med. 2014 Jan.-Feb.; 9(1):39-41.
15. Kanner J and Lapidot T. The stomach as a bioreactor: dietary lipid peroxidation in the gastric fluid and the effects of plant-derived antioxidants, Free Radical Biology and Medicine, Volume 31, Issue 11, 2001, Pages 1388-1395.

Acknowledgement of the above references herein is not to be inferred as meaning that these are in any way relevant to the patentability of the presently disclosed subject matter.

### BACKGROUND

Choline is an essential dietary component and its consumption is needed to maintain health, despite the fact that mammals can synthesize it in small amounts. Water soluble choline compounds such as phosphocholine (PCh), glycerophosphocholine (GPC) and choline (free choline and choline salt), serve a number of essential biological functions including preservation of the structural integrity of cell membranes, cell signaling, nerve impulse transmission, lipid (fat) transport and metabolism, and are also a source of methyl groups.

In 1998, the Food and Nutrition Board (FNB) of the Institute of Medicine set an Adequate Intake level for choline (Table 1). The main criteria for establishing the AI for choline was the prevention of liver damage.

TABLE 1
Adequate Intake (AI) for Choline

| Population | Age | AI (mg/day) | Upper limit (g/day) |
|---|---|---|---|
| Infants | 0-6 months | 125 | Not possible to establish. |
| | | ≅18 mg/kg | Source of intake should |
| | 7-12 months | 150 | be formula and food only |
| | | ≅17 mg/kg | |
| Children | 1-3 years | 200 | 1 |
| | 4-8 years | 250 | 1 |
| | 9-13 years | 375 | 2 |
| Males | 14-18 years | 550 | 3 |
| | ≧19 | 550 | 3.5 |
| Females | 14-18 years | 400 | 3 |
| | ≧19 | 425 | 3.5 |
| Pregnancy | All ages | 450 | Age-appropriate UL |
| Lactation | All ages | 550 | Age-appropriate UL |

The current available commercial sources of GPC, PCh and choline are either naturally occurring or synthetically made. Naturally occurring sources have the significant drawback of containing highly variable and minimal levels of GPC, choline and PCh (see USDA Database for the Choline Content of Common Foods, 2008) and are thus not suitable for dietary supplementation.

Therefore, the only available sources of GPC, choline and PCh that appear in controlled, concentrated and purified form are from synthetic sources. The synthetic sources of choline, GPC and PCh are formed by chemical or enzymatic processes from different starting materials and usually involve the use of either undesirable starting materials or catalysts that may limit the use of those synthetic products for food application, especially for infant nutrition.

Beside the limitation of using those synthetic products, previous data showed that the phosphate-esters compounds—including PCh and GPC—are potentially vulnerable compounds, and thus expected to be unstable during exposure to high temperature, oxygen and water. In addition, studies have shown that metal ions neutralize the negative charge on the phosphate, making it more susceptible to nucleophilic attack. The metal ions might also be able to accelerate the rate of phosphate ester hydrolysis by any or all of the following, again without wishing to be limited by the following: (a) stabilizing the leaving group (RO—) by coordination, (b) providing an effective OH— nucleophile at physiological pH and (c) organizing the reactants H₂O and ROPO₃⁻² to make the reaction effectively intramolecular. For example, during the production process, infant formulas are subjected to an environment that includes all of these problematic, risky parameters for PCh and GPC compounds—hydration, high temperature, oxygen, and metal ions. Therefore, these compounds are at increased risk of degradation if added to infant formulas.

Data collected from human milk demonstrate that the water soluble choline compounds concentrations are inconsistent in regards with the ratio between the different choline compounds. While some studies demonstrated that PCh is the major compound of choline followed by GPC, others demonstrated the opposite. Although PCh and GPC are the most prevalent choline compounds in human milk, no recommendations or regulations have been defined regarding supplementation of these water soluble choline compounds. Infant formulas contain significantly high levels of choline compared to human milk since they are usually supplemented only by choline salts—particularly choline bitartrate and choline chloride.

Choline supplementation might promote adverse effects since several studies demonstrated that choline is metabolized by gut flora, forming the metabolites: Trimethylamine (TMA) and Trimethylamine N-oxide (TMAO). Those molecules were shown to predict risk for CVD in an independent large clinical cohort and to promote up-regulation of multiple macrophage scavenger receptors linked to atherosclerosis and CVD risk.

### GENERAL DESCRIPTION

In the first aspect of the present invention there is provided a composition comprising at least one water soluble choline compound selected from a group consisting of GPC, PCh, and choline; wherein said at least one water soluble choline compound is derived from at least one natural source, and wherein the concentration of said at least one water soluble choline compound is at least 0.5% w/w of the composition.

The term “w/w percentage” or “% w/w” refers to weight percentage out of dry matter weight.

The term “water soluble choline compound” refers to any choline derivative that is soluble in water, such as for example compounds including choline (including both free choline and choline salt (e.g choline chloride, choline bitartarate and choline citrate)), PCh, GPC and any derivatives thereof.

The term “at least one water soluble choline compound”, refers to a single water soluble choline compound or any combination of water soluble choline compound derivatives as noted above. Therefore, at least one water soluble choline compound may refer to one water soluble choline compound being selected from choline, PCh, GPC; or to two water soluble choline compounds (choline and PCh or choline and GPC or GPC and PCh); or to three water soluble choline compounds (choline, PCh and GPC).

Throughout the invention, it is important to note that the term “composition” encompasses any type of pharmaceutical, nutraceutical, food composition or supplement for administration and metabolization by a subject that is produced by industrial means and which may at some embodiments be derived from natural sources, however is not a natural product and cannot be understood to encompass any naturally occurring composition such as for example human milk.

As noted in the first aspect of the invention the concentration of said at least one water soluble choline compound, whether it is a single compound or a combination of water soluble choline compound derivatives is at least 0.5% w/w of the composition. In some embodiments said at least one water soluble choline compound is at least 1% w/w of the composition, in other embodiments at least 3% w/w of the composition, in further embodiments 5% w/w of the composition, in yet further embodiments at least 10% w/w of the composition and in other embodiments at least 20% of the composition.

In some other embodiments, said composition comprises a combination of at least two water soluble choline compounds selected from a group consisting of GPC, PCh, choline or any combination thereof (i.e. any one of the combinations choline and GPC; choline and PCh; GPC and PCh). In other embodiments, said composition comprises, a combination of three water soluble choline compounds consisting of GPC, PCh and choline.

In some embodiments, wherein said water soluble choline compound comprises PCh, its concentration is at least 0.2% w/w of the composition. In some embodiments the concentration of PCh in a composition of the invention is at least 0.5% w/w of the composition, in other embodiments at least 1% w/w of the composition, in yet other embodiments at least 2% w/w of the composition and in further embodiments at least 5% w/w of the composition.

In some other embodiments, wherein said water soluble choline compound comprises GPC, its concentration is at least 0.5% w/w of the composition. In some embodiments, the concentration of GPC in a composition of the invention is at least 1% w/w of the composition, in other embodiments at least 3% w/w of the composition, in further embodiments 5% w/w of the composition, in other embodiments at least 10% w/w of the composition and yet further embodiments at least 20% w/w of the composition.

In further embodiments, wherein said composition comprises, as said water soluble choline compound, a combination of two or more water soluble choline compounds, wherein one of the water soluble choline compounds is GPC, said GPC constitutes at least 20% w/w of said water soluble choline compound. In other embodiments, GPC comprises between about 20% w/w to about 70% w/w of the water soluble choline compound. In other embodiments. GPC comprises between about 30% w/w to about 60% w/w of the water soluble choline compound and in yet further embodiments between about 40% w/w to about 50% w/w of the water soluble choline compound.

In some embodiments, wherein said composition comprises, as said water soluble choline compound, a combination of PCh and choline (in some other embodiments GPC, PCh and choline together), PCh and choline together constitute at least 1% w/w of the water soluble choline compound. In further embodiments, PCh and choline comprise between about 1% w/w to about 60% w/w of the water soluble choline compound. In other embodiments, PCh and choline comprise between about 10% w/w to about 50% w/w of the water soluble choline compound and in yet further embodiments between about 20% w/w to about 40% w/w of the water soluble choline compound.

In some embodiments, wherein said composition comprises, as said water soluble choline compound, a combination of GPC and PCh and optionally choline or a combination of GPC and choline and optionally PCh, PCh and choline together constitute at least 1% w/w of the water soluble choline compound. In further embodiments, PCh and choline comprise between about 1% w/w to about 60% w/w of the water soluble choline compound. In other embodiments, PCh and choline comprises between about 10% w/w to about 50% w/w of the water soluble choline compound and in yet further embodiments between about 20% w/w to about 40% w/w of the water soluble choline compound.

When referring to the fact that said at least one water soluble choline compound is “derived from natural source” it should be understood to encompass that said water soluble choline compound originated from a natural source, i.e. not a synthetic source. In some embodiments, said at least one natural source is selected from a group consisting of vegetable source, mammalian milk, animal source, egg, marine source, microorganism or aquaculture organisms and any combination thereof.

In some embodiments the natural source comprises mammalian milk (bovine milk, goat milk, sheep milk, buffalo milk and the like); in some embodiments the natural source comprises bovine milk.

The term “natural source” may also include any common and known product or food derived from the source (e.g. whey protein derived from bovine milk, skimmed bovine milk powder etc.). By common foods it is meant materials that are commonly eaten as foodstuffs. However, such common foods may optionally contain other substances added to them during preparation. For example, whey protein may contain higher levels of NaCl added during the cheese preparation process.

In some embodiments, said composition comprises at most 1 ppm of at least one of TMA, TMAO, ethylene oxide, glycidol and any combination thereof. In other embodiments said composition comprises at most 10 ppm of at least one of TMA, TMAO, ethylene oxide, glycidol and any combination thereof. said composition comprises at most 20 ppm of at least one of TMA, TMAO, ethylene oxide, glycidol and any combination thereof and in yet further embodiments at most 50 ppm.

Thus, the present invention, in at least some embodiments, overcomes the drawhacks of the prior art by providing compositions containing concentrated water soluble choline compounds that are purified from natural, non-synthetic sources and are therefore free of precursors used for synthetic preparations (e.g. ethylene oxide, glycidol, TMA, etc.) and are therefore also free from substances required to synthesize such preparations, such as catalysts (either chemical or enzymatic) that may create harmful by-products or remain in residual amounts in the final product.

In a further aspect the invention provides a process for the preparation of a composition comprising at least one water soluble choline compound; wherein said at least one water soluble choline compound is derived from at least one natural source, and wherein said concentration of said at least one water soluble choline compound is at least 0.5% w/w of the composition; said process comprising the steps of:
(i) providing at least one natural source of choline;
(ii) purifying said at least one natural source of choline.

Existing natural sources of water soluble choline compounds contain very low and highly variable concentrations of the compounds. Therefore, it is not commercially practical to use them for administration of water soluble choline compounds.

The inventors have demonstrated that technologies intended for removal of contaminants that are a minority portion of the mixture (for example, less than 50% of the mixture) are also efficient for purifying those substances from very low residual levels, removing above 50% of the starting material, preferably above 80% or even 90% of the mixture.

In some embodiments of a process of the invention said at least one natural source of choline contains less than 0.5% w/w GPC, 0.5% w/w PCh and/or 0.5% w/w choline. In further embodiment of a process of the invention said at least one natural source of choline contains at most 1% w/w GPC, 0.5% w/w PCh and/or 0.5% w/w choline. In further embodiments, said at least one natural source of choline contains at most 0.4% w/w GPC, 0.4% w/w PCh and/or 0.4% w/w choline. In other embodiments, said at least one natural source of choline contains at most 0.3% w/w GPC, 0.3% w/w PCh and/or 0.3% w/w choline. In other embodiments, said at least one natural source of choline contains at most 0.2% w/w GPC, 0.2% w/w PCh and/or 0.2% w/w choline.

In further embodiments of a process of the invention said purification include, but are not limited to, extraction, crystallization, chromatography, ion exchange purification, membrane purification, ultra filtration, nano filtration, micro filtration, electrodialysis or water washes.

In another embodiment a process of the invention further comprises the step of (iii) extracting said natural source with an organic solvent comprising an alcohol of 1 to 4 carbon atoms.

In another embodiment the process result in a composition with controllable concentration of said water soluble choline compound. By “controllable” it is meant that the concentration may be increased or decreased, for example optionally according to the needs of the composition or its final application (such as ingestion by a subject).

In another one of its aspects the invention provides a composition comprising at least two water soluble choline compounds selected from a group consisting of GPC, PCh and choline; wherein said at least two water soluble choline compounds comprise at least 0.5% w/w of the composition; and wherein PCh and choline together comprise at least 1% w/w of said water soluble choline compounds.

It is noted that in case the composition does not include one of PCh or choline their concentration is null. Thus, in some embodiments, the term “PCh and choline” refer to the two water soluble choline compounds, in other embodiments in case PCh is not part of the water soluble choline compounds of the composition the term relates only to choline; in yet further embodiments in case choline is not part of the water soluble choline compounds of the composition the term relates only to PCh.

In some embodiments, the composition comprises GPC, PCh and choline.

In some embodiments, said water soluble choline compounds comprise between about 1% w/w to 100% w/w of said composition. In other embodiments, said water soluble choline compounds comprise at least 1% w/w of the composition. In other embodiments said water soluble choline compound comprise at least 3% w/w of the composition, in further embodiments 5% w/w of the composition, in yet further embodiments at least 10% w/w of the composition and in other embodiments at least 20% of the composition.

In some embodiments, PCh concentration is at least 0.2% w/w of the composition. In some embodiments the concentration of PCh in a composition of the invention is at least 0.5% w/w of the composition, in other embodiments at least 1% w/w of the composition, in yet other embodiments at least 2% w/w of the composition and in further embodiments at least 5% w/w of the composition.

In some other embodiments, GPC concentration is at least 0.5% w/w of the composition. In some embodiments, the concentration of GPC in a composition of the invention is at least 1% w/w of the composition, in other embodiments at least 3% w/w of the composition, in further embodiments 5% w/w of the composition, in other embodiments at least 10% w/w of the composition and yet further embodiments at least 20% w/w of the composition.

In other embodiments, said GPC comprises at least 20% w/w of said water soluble choline compounds. In other embodiments, said GPC comprises between 20% w/w to about 70% w/w of said water soluble choline compounds. In other embodiments, said GPC comprises between about 30% w/w to about 60% w/w of the water soluble choline compound and in yet further embodiments between about 40% w/w to about 50% w/w of the water soluble choline compound.

In some embodiments, said PCh comprises between about 1% w/w to 70% w/w of said water soluble choline compounds. In other embodiments, said PCh comprises between about 20% w/w to about 60% w/w of the water soluble choline compound and in yet further embodiments between about 30% w/w to about 40% w/w of the water soluble choline compound.

In other embodiments, said choline comprises between about 1% w/w to 25% w/w of said water soluble choline compounds. In other embodiments, said choline comprises between about 5% w/w to about 20% w/w of the water soluble choline compound and in yet further embodiments between about 10% w/w to about 15% w/w of the water soluble choline compound.

It should be understood that under this aspect, said water soluble choline compounds are derived from any available source of choline or its components. In some embodiments, at least one of said GPC, PCh or choline is derived from a natural source. In other embodiments, said at least one of said GPC, PCh or choline is derived from a synthetic source.

In some embodiments, said composition comprises at most 1 ppm of at least one of TMA, TMAO, ethylene oxide, glycidol and any combination thereof. In other embodiments, said composition comprises at most 10 ppm of at least one of TMA, TMAO, ethylene oxide, glycidol and any combination thereof. In other embodiments said composition comprises at most 20 ppm of at least one of TMA, TMAO, ethylene oxide, glycidol and any combination thereof. said composition comprises at most 30 ppm of at least one of TMA, TMAO, ethylene oxide, glycidol and any combination thereof and in yet further embodiments at most 50 ppm.

In a further aspect the invention provides a composition comprising GPC and PCh; wherein the molar concentration of PCh is greater than the molar concentration of GPC.

In a further aspect the invention provides a composition comprising GPC and PCh; wherein the molar concentration of PCh equals the molar concentration of GPC.

In some embodiments, GPC and PCh comprise at least 0.05% w/w of the composition.

In some embodiments, said GPC and PCh comprise at least 0.1% w/w of the composition; in other embodiments at least 0.5% w/w of the composition; in further embodiments at least 1% w/w of the composition, in other embodiments at least 2% w/w of the composition and in further embodiments at least 5% w/w of the composition. In other embodiments at least 10% w/w of the composition and in further embodiments at least 20% w/w of the composition.

In further embodiments, said composition further comprises choline wherein GPC, PCh and choline comprise at least 0.05% w/w of the composition.

In some embodiments, said GPC, PCh and choline comprise at least 0.1% w/w of the composition; in other embodiments at least 0.5% w/w of the composition; in further embodiments at least 1% w/w of the composition, in other embodiments at least 2% w/w of the composition and in further embodiments at least 5% w/w of the composition. In other embodiments at least 10% w/w of the composition and in further embodiments at least 20% w/w of the composition.

In some further embodiments, said composition is capable of being chemically stable at storage temperatures of 20-30° C. for at least 12 months. In other embodiments said composition is capable of being chemically stable at storage temperatures of 23-27° C. for at least 12 months. In other embodiments said composition is capable of being chemically stable at storage temperatures of 25° C. for at least 12 months.

In another embodiment GPC and PCh levels are stable at up to 42° C. for at least 3 months with not more than 20% degradation. In a further embodiment GPC and PCh levels are stable at 38-42° C. for at least 3 months. Optionally the levels are stable for at least 4 months. Optionally the levels are stable for up to 6 months.

In a further embodiment, said stability refers to a degradation level of at least one water soluble choline compound of less than 20% w/w. In other embodiments said composition has a degradation level of at least one water soluble choline compound of less than 15% w/w. In further embodiments said composition has a degradation level of at least one water soluble choline compound of less than 10% w/w and in yet another embodiment of less than 5%.

In a further embodiment, said stability refers to a degradation level of GPC and/or PCh of 20% w/w or less. In other embodiments said composition has a degradation level of GPC and/or PCh of 15% w/w or less. In further embodiments said composition has a degradation level of GPC and/or PCh of 10% w/w or less and in yet another embodiment of 5% or less, preferably 1% or less.

In some further embodiments, the molar concentration of GPC is greater than the molar concentration of choline (i.e. [PCh]>[GPC]>[choline]). In other embodiments, the molar concentration of choline is greater than the molar concentration of GPC and lower than the molar concentration of PCh (i.e. [PCh]>[choline]>[GPC]). In yet other embodiments, the molar concentration of choline is greater than the molar concentration of PCh (i.e. [choline]>[PCh]>[GPC]). In further embodiments, the molar concentration of PCh is greater than the molar concentration of choline.

In further embodiments, the weight ratio of PCh to GPC is at least about 0.70. In some embodiments, the weight ratio of PCh to GPC is at least about 0.8. In other embodiments, the weight ratio of PCh to GPC is at least about 1. In further embodiments, the weight ratio of PCh to GPC is at least about 1.1 or 1.2. In other embodiments at least 1.5, in other embodiments at least 2 and in yet another embodiment at least about 3.

In some embodiments of the invention a composition further comprises betaine.

In other embodiments, said composition of the invention is formulated to a pharmaceutical, a dietary supplement, a medical food a nutritional or a neutraceutical composition.

In another aspect the invention provides a composition of the invention as described hereinabove for use in the preparation of a pharmaceutical, a dietary supplement, a medical food, a nutritional or a neutraceutical composition.

In another aspect the invention provides a pharmaceutical, a dietary supplement, a medical food a nutritional or a neutraceutical composition comprising a composition of the invention.

A medical food as used herein is any food product that has been formulated and intended for the dietary management of a subject suffering from a disease, disorder or condition that has distinctive nutritional needs which are difficult to meet with normal diet alone.

In certain other non-limiting embodiments of the present invention, the composition of the invention is formulated into a food product or a dietary supplement selected from a biscuit, pastry, cake, bread, cereal, bar, snack, pill, tablet, pellets, dragees, capsule, soft gel, syrup, infant formula, baby formula, toddler food, adult formula, medical nutrition product, candy, gummy, or confectionary.

Pharmaceutical compositions and dietary supplements suitable for oral administration may be presented as discrete dosage units such as pills, tablets, capsules, or as a powder or granules, or as a solution or suspension.

The invention provides a formula comprising a composition of the invention. In some embodiments, said formula further comprising at least one of a physiologically acceptable lipid, protein, carbohydrate, vitamin, mineral, amino acid, nucleotide and active or non-active additive. In some embodiments said formula is an infant formula. In some embodiments said formula is a follow on formula or a toddler formula. In some embodiments said formula is a child formula. In some embodiments said formula is an adult formula.

According to at least some embodiments, there is provided a method for producing pharmaceutical or nutritional composition as described herein comprising a spray drying process wherein the water soluble choline compounds maintain their stability.

According to some embodiments of the present invention, during formula production, the compositions of the invention are added with all other minerals and vitamins prior to homogenization and spray drying or by other methods.

In some embodiments, said lipid comprises one or more of palm and palm kernel oils, soybean oil, palm olein, coconut oil, canola oil, olive oil, cottonseed oils, medium chain triglyceride (MCT) oil, sunflower oil, high oleic sunflower oil, safflower oil, high oleic safflower oil, algal oil, marine oils and combinations thereof; wherein said protein comprises hydrolyzed, partially hydrolyzed, non-hydrolyzed or intact proteins, and any combinations thereof; wherein amino acids are selected from the group consisting of alanine, arginine, asparagine, carnitine, aspartic acid, cystine, glutamic acid, glutamine, glycine, histidine, isoleucine, leucine, lysine, methionine, phenylalanine, proline, serine, taurine, threonine, tryptophan, taurine, tyrosine, valine, and combinations thereof; wherein the carbohydrate comprises one or more of hydrolyzed, intact, naturally and/or chemically modified cornstarch, maltodextrin, glucose polymers, sucrose, corn syrup, corn syrup solids, rice or potato derived carbohydrate, glucose, fructose, lactose, high fructose corn syrup and indigestible oligosaccharides such as fructooligosaccharides (FOS), galactooligosaccharides (GOS), and combinations thereof.

According to at least some embodiments, there is provided a method for improving, promoting or maintaining the development of phospholipid synthesis or lipoprotein synthesis in a subject comprising administering a composition of the invention as described herein.

According to at least some embodiments, there is provided a method for improving, promoting or maintaining proper sulphur amino acid metabolism, in a subject comprising administering a composition of the invention as described herein.

According to at least some embodiments, there is provided a method for improving, promoting or maintaining choline plasma levels in a subject comprising administering a composition of the invention as described herein.

According to at least some embodiments, there is provided a method for improving, promoting or maintaining enhancement of osmo-regulation in a subject comprising administering a composition of the invention as described herein.

In another aspect the invention provides a method for improving, promoting or maintaining plasma levels of one or both of growth hormone or ketone bodies in a subject comprising administering a composition of the invention.

In another aspect the invention provides a method for improving, promoting or maintaining intestinal absorption of minerals, trace elements, metals or vitamins in a subject comprising administering a composition of the invention.

In yet a further aspect the invention provides a method for improving, promoting or maintaining choline plasma levels in a subject comprising administering a composition of the invention.

In another aspect the invention provides a method for improving, promoting or maintaining cognitive functions in a subject comprising administering a composition of the invention.

In another aspect the invention provides a method for improving, promoting or maintaining mineral or metals absorption in a subject comprising administering a composition of the invention.

In another aspect the invention provides a method for improving, promoting or maintaining gut flora balance in a subject comprising administering a composition of the invention.

In a further aspect the invention provides a method for preventing or treating or improving or reducing symptoms of: Neurodegenerative diseases, Alzheimer's disease, Parkinson's disease, Amyotrophic lateral sclerosis (ALS), dementia, stroke, cognitive decline, chemotherapy-related cognitive decline, malnutrition or unbalanced nutrition, insufficient oral food intake, liver disease, liver dysfunction, alcoholic liver disease or renal dysfunction, comprising administering a composition of the invention.

In a further aspect the invention provides a method of administering at least one water soluble choline compound to a subject comprising administering to said subject a composition of the invention, wherein upon administration of said composition the TMA or TMAO levels in the gut, plasma, urine, brain, liver and intestine or in the plasma of said subject are maintained or reduced.

In some embodiments of the invention said subject is an infant.

An infant as used herein is meant to encompass a human infant, including but not limited to, a newborn, a preterm and term infant, small premature infants, infants with very low birth weight (VLBW) or extreme low birth weight (ELBW) particularly those with general immaturity, for example of the gastrointestinal track or any other health risks known to a person skilled in the art. In some embodiments of the invention said subject is a toddler. In some embodiments of the invention said subject is a child. In other embodiments, said subject is an adult (including, a male, a female in child bearing age pre or post gestation, a teenager, an elderly senior subject). In other embodiments, said subject is a pregnant or lactating woman.

### DETAILED DESCRIPTION OF EMBODIMENTS

### Example 1

### Production of Compositions According to the Invention

### A. Extraction and Concentration of Water Soluble Choline Compounds Using Ethanol as a Solvent to Obtain a Powder Product.

Whey stream from dairy production was concentrated by evaporation of water and then crystalized to yield lactose crystals that were separated by filtration from their mother liquor. Mother liquor was partially demineralized by nanofiltration membrane and dried by means of spray drying to obtain dry powder.

Ten grams of the dried powder were mixed with 200 ml of ethanol 90% (with 10% water) for 2 hours at 40° to obtain slurry. The slurry was filtered through Buchner filter in order to separate between the filtrate and the solids. The filtrate was evaporated, under reduced pressure, using rotary evaporator to obtain dry powder. The dry powder obtained contained 5.5% GPC and 0.52% PCh (as determined by ³¹P-NMR).

### B. GPC Enrichment Using Ion Exchange Columns.

Ten grams of the final product from Example 1A (containing 5.5% GPC and 0.52% PCh) were dissolved in 40 ml of water and then were passed through a glass column containing 100 ml of strong anionic exchange resin (Doc2001). The solution coming out of the first column was then transferred through a strong cationic resin (001×7) column and was neutralized by a weak anionic resin (D301) column, in order to remove minerals and to obtain a neutralized filtrate. All resins were obtained from JIANGSU SUQING WATER TREATMENT ENGINEERING GROUP CO. Finally, the neutralized filtrate was evaporated using a rotary evaporator, under reduced pressure, to receive a product containing 20.5% GPC and 0.03% of PCh (as determined by ³¹P-NMR).

### C. GPC Enrichment Using a Silica Chromatography Procedure

Ten grams of the final product of Example 1A (containing 5.5% GPC) were dissolved in 20 ml of ethanol:water (80%:20% v/v) solution and were loaded on a silica gel chromatography column filled with 70 grams of Davisil “LC60A 20-45 μm” silica from the Grace Company. 500 ml ethanol:water (80%:20% v/v) was then transferred through the column in order to separate a lactose containing fraction from a GPC containing fraction. After the first 200 ml of the ethanol containing solvent was applied to the column, the collection of GPC containing fractions of the filtrate commenced. The GPC containing fractions were then evaporated using rotary evaporator, under reduced pressure, to obtain a dry powder. The dried powder obtained was injected to the HPLC with ELS detector against a sample of the product of Example 1A, in the same concentration. The relative peak area of the GPC was about 5 fold higher in the purified product compared to the product of example 1A.

D. Purification of Choline Compounds from Whey Stream with Methanol as a Solvent to Obtain a Powder Product.

Whey stream from dairy production was used to produce whey protein concentrate by diafiltration using Ultra filtration membranes. The permeate from the membranes was demineralized by means of electrodialysis. The mineral free stream was dried by spray dryer to produce a powder. Five grams of this dried powder were mixed with 40 ml of methanol for 2 hours at 25° C. The whole sample was then centrifuged for 5 minutes at 6,000 RPM in order to separate between the solution and the solids. The solution was evaporated using rotary evaporator, under reduced pressure, to receive dry powder. The dry powder obtained contained 7.6% GPC and 0.4% PCh (By ³¹P-NMR).

E. Purification of GPC from “Lactosalt Optitase”

Dairy salts fraction called “Lactosalt Optitase” (Armor) containing about 85% salts, 5% moisture and 0.5% protein was purified by electrodialysis. Purification was performed using a PCCell ED 64-4 Electrodialysis cell unit. This unit has a 10 parallel cell pair stack structure. The active size of each membrane is 8×8 cm (active area of 0.0064 m2). Hence, total active area is 0.064 m².

For the electrolyte circuit, a 0.25M solution of sodium sulfate was used. The anolyte and the catholyte chambers were connected in series. A circulating NaCl solution served as the concentrate. Its initial concentration was around 1000 mg/lit. 1 liter of solution containing 10 gr of “Lactosalt Optitase” dissolved in demineralized water was fed to the circulating chamber. The voltage was pre-set at its highest value (36.5 Volts for the stack). Recirculation was stopped when further significant decreases in conductivity were no longer noted in the salts solution. Sample of the purified solution was dried by rotary evaporator, under reduced pressure, to dryness. The dried product obtained was injected to the HPLC with ELS detector against a sample of the same concentration of the original “Lactosalt Optitase”. The relative peak area of the GPC was about 10 fold higher in the purified product compared to the raw material.

F. Purifcation of GPC from Krill Meal

200 gr. of Superba Krill meal were mixed together with 1 liter of methanol for one hour at 25° C. The whole solution was then filtered through Buchner filter in order to separate between the filtrate and the solids. The filtrate was evaporated using rotary evaporator to obtain 35 gr of oil. Thirty five ml of purified water and thirty five ml of butanol were added to the oil and the solution was mixed for few minutes. Phase separation was achieved in a separatory funnel. The bottom phase was evaporated to obtain 2.78 gr. which contained 7.1% GPC (by ³¹P-NMR).

### G. Preparation of Gummy Bears Containing GPC

0.4 gr of Citric acid and 0.37 gr of Trisodium citrate were dissolved in 30 ml water using agitation. The solution was heated up to 75° C., followed by the addition of 5 gr of white sugar (Sucrose) and 1.5 gr of Citrus Pectin. The mixture was heated up to 100° C., and agitated at 100° C. for 2-3 minutes. 30 gr of glucose syrup 80% and 50 gr white sugar (Sucrose) were added and the mixture was heated up to 108° C. under continuous agitation until full dissolution and 78°Bx is achieved (about 40-50 minutes). The solution was cooled down to 100° C. and 1.06 gr product of example No. 1A were added. Agitation was continued at 100° C. for 2-3 minutes, and the following flavorings and colorings were added: 0.5 gr Adipic acid, 0.15 gr strawberry flavor essence, 0.5 ml Lemon juice, 0.15 gr shade ruby red essence, 0.77 gr Citric acid 50%. Agitation was continued at 100° C. for 2-3 more minutes, than heat source was stopped and product obtained was poured into molds at 90-100° C. Molds were placed in an air conditioned room for about 48 hours for drying.

### Example 2

### GPC Stability of Natural Vs. Synthetic Choline Composition in Stomach Model

More than 90% of orally administrated GPC is absorbed from the intestine. Once absorbed, GPC is rapidly circulated to all organs and taken up into the cells. It is thus desirable that GPC will be minimally affected by gastrointestinal conditions and remain intact without any modifications which might affect its activity and efficacy.

In order to test the gastrointestinal stability of natural water soluble choline compounds according to the invention, in comparison with synthetic, conventional choline compounds an in vitro gastric (stomach) model was used.

In vitro gastric model was conducted as previously described by Kanner and Lapidot 2001 using simulated gastric fluid (SGF) as an artificial dissolution medium intended to represent stomach conditions. SGF was prepared according to the U.S. Pharmacopoeia by dissolving 0.2% w/w sodium chloride and 0.32% w/w of purified pepsin (derived from porcine stomach mucosa) in acidic water, pH about 1.2.

Choline compounds were purified from mother liquor from lactose crystallization. The purification included two stages: first stage of membrane purification and a second stage of crystallization.

Synthetic water soluble choline compound was produced from soy lecithin by a reaction using Sodium methoxide as a catalyst to obtain GPC and methyl esters. Several purification steps were performed in order to separate between the methyl esters and GPC. In addition, a mineral removal step was performed using ion exchangers.

Both natural and synthetic water soluble choline compounds (compositions are detailed in Table 2) were incubated with SGF at 37° C. in a shaking bath for 180 minutes and then the samples were analyzed for GPC levels by HPLC.

The results, presented in table 2, show that a composition containing about 5% w/w of natural, water soluble, choline compound from dairy source did not demonstrate any GPC degradation (GPC level remained constant) following 180 minutes incubation in gastric model conditions. On the other hand, the composition which contained synthetic, water soluble, choline compound, demonstrated degradation of about 5.4% of the GPC (from 30.50 mg GPC to 28.84 mg GPC).

These results demonstrate that compositions containing water soluble choline compounds according to the invention are more stable in gastric conditions than other compositions which contain conventional choline compounds.

TABLE 2

| Composition | A | | B | |
|---|---|---|---|---|
| Source of water | Natural (Dairy) | | Synthetic | |
| soluble choline | | | | |
| compound | | | | |
| % PCh and Choline | 12.5% | | <1% | |
| from total choline | | | | |
| compounds (w/w) at | | | | |
| baseline | | | | |
| | | after | | after |
| | at time 0 | 180 min | at time 0 | 180 min |
| GPC (mg GPC/ml | 35.35 | 35.48 | 30.50 | 28.84 |
| SGF) | | | | |

| | | |
|---|---|---|
| % GPC Degradation | 0 | 5.4 |

### Example 3

### The Effect of Different Water Soluble Choline Compounds on the Bioavailability of Choline

Study Design

The bioavailability of different water soluble choline compounds was investigated in an animal model of neonatal Sprague Dawley rats aged 3-5 days. Animals were randomly assigned to one of the three diets, twelve rats per group. Animals within a litter were randomly assigned across treatments.

The study groups were:

Group A: Formula Containing Choline

Choline chloride was purchased from Sigma Chemical Company

Group B: Formula Containing GPC and Phosphocholine (Phosphocholine>GPC)

Synthetic water soluble choline compound was produced from soy lecithin by a reaction using sodium methoxide as a catalyst to obtain GPC and methyl esters. Several purification steps were performed in order to separate between the methyl esters and GPC. In addition, a mineral removal step was performed using ion exchangers. Phosphocholine chloride calcium salt tetrahydrate was purchased from Sigma Chemical Company

Group C: Formula Containing GPC (GPC>Phosphocholine)

Choline compounds were purified from mother liquor from lactose crystallization. The purification included two stages: Ion exchange purification using a strong cationic resin (001×7) column and weak anionic resin (D301) column (all resins were obtained from JIANGSU SUQING WATER TREATMENT ENGINEERING GROUP CO.) and chromatographic purification based on UBK535K resin from Diaion company.

All diets contained the same added choline equivalent levels that originated from different water soluble choline compounds. The water soluble choline compound composition of each diet is detailed in table 3.

TABLE 3

| | Group A | Group B | Group C |
|---|---|---|---|
| Source of the choline | synthetic | synthetic | natural |
| compound | | | |
| Total choline equivalent | 679 | 680 | 679 |
| mg/liter formula | | | |
| Choline mg/liter formula | 678.4 | NA | NA |
| Choline microMolar | 6525 | NA | NA |
| GPC mg/liter formula | NA | 826.65 | 1676 |
| GPC microMolar | NA | 3217 | 6525 |
| Phosphocholine | NA | 610.4 | 0.67 |
| mg/liter formula | | | |
| Phosphocholine | NA | 3317 | 2 |
| microMolar | | | |
| Choline compound out of | 74.8 | 69 | 24.8 |
| composition % | | | |
| Choline + phosphocholine | 100 | 41.9 | <1 |
| out of choline compound % | | | |

NA = Not Applicable

Gastrostomy Tube Fed Infant Rats:

The gastrostomy tube fed rat pup is model to mimic infants fed formula, using tube feeding to overcome the difficulties in bottle-feeding of neonatal rats. The milk formulas were based on rat milk, with ingredients modified to meet the study objectives. The model enables complete control of the volume and thus nutrient intake. This avoids any difficulties due to variable intake across treatment groups. The animals were reared by milk feeding from 3-5 to 18-20 days of age. Milk volume is calculated daily based on the animal weight.

Plasma and Tissue Collection:

Blood samples were centrifuged at 2000 g×10 minutes, and plasma was recovered. In order to standardize tissue sample location from every animal, tissue samples harvest was done following the same protocol.

List of Analyses:

In plasma: phosphatidycholine, cholesterol, triglycerides, TMA/TMAO, free choline, glycerophosphocholine, phosphocholine, betaine. DMG, homocysteine, methionine, cysteine, VLDL+LDL, HDL cholesterol, TAG, Growth hormone, Insulin growth factor 1 (IGF-1), Keto bodies, Folate.

In urine: TMA/TMAO.
In liver: S adenosyl methionine and S adenosyl homocysteine, triglyceride.

Results

Plasma samples were analyzed for choline level. The results are presented in Table 4 and demonstrate better bioavailability of choline in group B in comparison with group A.

TABLE 4

| | Choline (uM in plasma) |
|---|---|
| Group A | 10.8 ± 1.2 |
| Group B | 12.3 ± 0.8 |
| Group C | |

In addition, in comparison with group A, groups B and C have: lower TMA and TMAO levels in plasma and urine, increased Phosphatidylcholine synthesis, phospholipids/triglycerides ratio and absorption of important lipids (e.g cholesterol, fat soluble vitamins, hormones and carotenoids), reduced chylomicrons particle size in comparison, improved plasma lipids profile, higher methionine levels and lower homocysteine levels in liver and plasma, increased growth hormone secretion, fatty liver hepatic oxidation, ketone bodies levels and minerals and metals absorption. These are associated with lower risk to develop CVD and atherosclerosis and improved cognitive abilities and central nervous system (CNS) functions.

### CONCLUSION

The above results demonstrate that animals fed formula which contained the composition of the invention (Group B) had better bioavailability of choline in comparison with animals that were fed the control formula (Group A).

### Example 4

### GPC Stability in Infant Formula Containing Water Soluble Choline Compounds According to the Invention in Comparison with Infant Formula Containing Conventional Water Soluble Choline Compounds

Two types of infant formulas were prepared in pilot scale by the following method: Skimmed milk powder, lactose and concentrated whey protein (80%) were mixed into distilled water by a high speed agitator and warmed to 65-70° C. Following 5 minutes of mixing, different recipes of waters soluble choline compounds, minerals, nucleotides, amino acids and vitamins were added. Following an additional 15 minutes, an oil mixture containing vegetable oils including ARA (Arachidonic acid) oil and DHA (Docosahexaenoic acid) oil were added. Mixing continued for additional 15 minutes. Then, the mixture was homogenized by “APV Rannie pressure homogenizer” with two-stage assembly: 70 Bars at stage 1 and 240 Bars at stage 2. Next, the homogenized mixture was spray dried by typical “Spray Dryer” at a rate of 20 liter/hr with air inlet temp of about 180° c. and air outlet temp of about 80° c. Dried powder was collected and dry blended with a premix (about 0.37%) of minerals and elemental substances.

At the end of this stage the two formulas contained a different composition of choline compounds: while 348-80-1 contained a molar ratio of GPC>PCh, formula no. 348-80-6 contained a molar ratio of GPC<PCh.

100 gr aliquots from each formula were packed under nitrogen environment in sealed airtight aluminum packages and stored at 25° C.±2 (Humidity 60%±5%) in temperature and moisture controlled storage chambers.

At baseline and following 12 months of storage, samples were analyzed for GPC and PCh content.

Table 5 shows stability results following 12 months at 25° C.±2 (Humidity 60%±5%). Sample 348-80-6, containing PCh>GPC ratio demonstrated minor GPC degradation level (about 4%) while formula 348-80-1, containing GPC>PCh ratio, resulted in about 38% GPC degradation. These results demonstrate that compositions containing water soluble choline compounds according to the invention are more stable than other compositions which contain conventional choline compounds.

TABLE 5

| | Formula | |
|---|---|---|
| | 348-80-1 | 348-80-6 |
| GPC- Phosphocholine Ratio | GPC > | GPC < |
| | Phosphocholine | Phosphocholine |
| % w/w PCh and choline from | >1 | >1 |
| water soluble choline | | |
| compounds | | |
| % w/w Choline | 0.09 | 0.013 |
| % w/w PCh T0 | NA | 0.09 |
| % w/w PCh Following 12 | NA | 0.09 |
| month of storage at 25° C. ± 2 | | |
| % w/w GPC T0 | 0.037 | 0.024 |
| % w/w GPC Following 12 | 0.023 | 0.023 |
| month of storage at 25° C. ± 2 | | |
| % GPC degradation | 38 | 4.2 |
| Following 12 month of storage | | |
| at 25° C. ± 2 | | |
| % PCh degradation | NA | 0 |
| Following 12 month of storage | | |
| at 25° C. ± 2 | | |

NA = Not Applicable
T0 = baseline values

1. A composition comprising at least one water soluble choline compound selected from a group consisting of GPC, PCh and choline; wherein said at least one water soluble choline compound is purified from at least one natural source, and wherein the concentration of said at least one water soluble choline compound is at least 0.5% w/w of the composition.

2. A composition according to claim 1, wherein said at least one natural source is selected from a group consisting of vegetable source, mammalian milk, animal source, egg, marine source, microorganism or aquaculture organisms and any combination thereof.

3. (canceled)

4. The composition according to claim 2, wherein said at least one natural source is a mammalian milk.

5-6. (canceled)

7. The composition according to claim 1, wherein PCh comprises at least 0.2% w/w of the composition.

8. The composition according to claim 1, wherein GPC comprises at least 0.5% w/w of the composition.

9. (canceled)

10. The composition according to claim 8, wherein GPC comprises between 20% w/w to 70% w/w of the at least one water soluble choline compound.

11. (canceled)

12. The composition according to claim 1, wherein PCh and choline comprise between 1% w/w to 60% w/w of at least one water soluble choline compound.

13. The composition according to claim 1, comprising at most 50 ppm of at least one of TMA, TMAO, ethylene oxide, glycidol and any combination thereof.

14. The composition according to claim 1, wherein said composition is free of precursors used for synthetic preparations and/or from substances required to synthesize such preparations.

15. A process for the preparation of a composition according to claim 1, comprising the steps of (i) providing at least one natural source of choline; (ii) purifying said at least one natural source of choline.

16. (canceled)

17. The process according to claim 15, wherein said at least one natural source is a mammalian milk.

18. (canceled)

19. The process according to claim 15 wherein said at least one natural source of choline contains of less than 0.5% w/w GPC, 0.5% w/w PCh and/or 0.5% w/w choline.

20. (canceled)

21. The process according to claim 15, further comprising the step of (iii) extracting said natural source with an organic solvent comprising an alcohol of 1 to 4 carbon atoms.

22. A composition comprising at least two water soluble choline compounds selected from a group consisting of GPC, PCh and choline; wherein said at least two water soluble choline compounds comprise at least 0.5% w/w of the composition; wherein PCh comprises between 1% w/w to 70% w/w of said water soluble choline compounds; and wherein PCh and choline comprise at least 1% w/w of said water soluble choline compounds.

23. (canceled)

24. The composition according to claim 22, comprising GPC, PCh and choline.

25. (canceled)

26. The composition according to claim 22, wherein said GPC comprises between 20% w/w to 70% w/w of said water soluble choline compounds.

27. (canceled)

28. The composition according to claim 22, wherein at least one of said GPC, PCh or choline is derived from a natural source.

29. (canceled)

30. The composition according to claim 22, wherein the molar concentration of PCh is greater than the molar concentration of GPC.

31. A composition comprising GPC and PCh; wherein the molar concentration of PCh is equal or greater than the molar concentration of GPC.

32. The composition of claim 31 wherein the concentration of GPC and PCh is at least 0.05% w/w of the composition.

33-44. (canceled)

45. An infant formula comprising a composition according to claim 1.

46-47. (canceled)

48. A method comprising administering the composition according to claim 1 to a subject, wherein said method is for one or more of:
(i) improving, promoting or maintaining plasma levels of one or both of growth hormone or ketone bodies in said subject;
(ii) improving, promoting or maintaining choline plasma levels in said subject;
(iii) improving, promoting, or maintaining cognitive functions in said subject;
(iv) improving, promoting or maintaining mineral or metals absorption in said subject; and
(v) for preventing, treating or improving or reducing symptoms of: Neurodegenerative diseases, Alzheimer's disease, Parkinson's disease, Amyotrophic lateral sclerosis (ALS), dementia, stroke, cognitive decline, chemotherapy-related cognitive decline, malnutrition or unbalanced nutrition, insufficient oral food intake liver disease, liver dysfunction, alcoholic liver disease or renal dysfunction in said subject.

49-55. (canceled)

56. An infant formula comprising a composition according to claim 22.

57. An infant formula comprising a composition according to claim 31.

58. A method comprising administering the composition according to claim 22 to a subject, wherein said method is for one or more of:
(i) improving, promoting or maintaining plasma levels of one or both of growth hormone or ketone bodies in said subject;
(ii) improving, promoting or maintaining choline plasma levels in said subject;
(iii) improving, promoting, or maintaining cognitive functions in said subject;
(iv) improving, promoting or maintaining mineral or metals absorption in said subject; and
(v) for preventing, treating or improving or reducing symptoms of: Neurodegenerative diseases, Alzheimer's disease, Parkinson's disease, Amyotrophic lateral sclerosis (ALS), dementia, stroke, cognitive decline, chemotherapy-related cognitive decline, malnutrition or unbalanced nutrition, insufficient oral food intake liver disease, liver dysfunction, alcoholic liver disease or renal dysfunction in said subject.

59. A method comprising administering the composition according to claim 31 to a subject, wherein said method is for one or more of:
(i) improving, promoting or maintaining plasma levels of one or both of growth hormone or ketone bodies in said subject;
(ii) improving, promoting or maintaining choline plasma levels in said subject;
(iii) improving, promoting, or maintaining cognitive functions in said subject;
(iv) improving, promoting or maintaining mineral or metals absorption in said subject; and
(v) for preventing, treating or improving or reducing symptoms of: Neurodegenerative diseases, Alzheimer's disease, Parkinson's disease, Amyotrophic lateral sclerosis (ALS), dementia, stroke, cognitive decline, chemotherapy-related cognitive decline, malnutrition or unbalanced nutrition, insufficient oral food intake liver disease, liver dysfunction, alcoholic liver disease or renal dysfunction in said subject.
